(19) United States
(12) Patent Application Publication
(10) Pub. No.: US 2024/0008979 A1
(43) Pub. Date: Jan. 11, 2024

(54) PROSTHETIC CARDIAC VALVE DELIVERY SYSTEMS AND METHODS

(71) Applicant: SHIFAMED HOLDINGS, LLC, Campbell, CA (US)

(72) Inventors: Connor MULCAHY, San Francisco, CA (US); Alice YANG, Campbell, CA (US); Claudio ARGENTO, Felton, CA (US)

(21) Appl. No.: 18/043,480

(22) PCT Filed: Aug. 30, 2021
(86) PCT No.: PCT/US2021/048126

§ 371 Date: Feb. 28, 2023

Related U.S. Application Data
(60) Provisional application No. 63/072,797, filed on Aug. 31, 2020.

(51) Int. Cl. A61F 2/24

(52) U.S. Cl. CPC A61F 2/2427; A61F 2/2457; A61F 2220/0016

(57) ABSTRACT

A method for treating a diseased native valve in a patient includes 5 advancing a distal end of a delivery device to a first side of a native valve, deploying an anchor from a delivery configuration to a deployed configuration on the first side of the native valve, and rotating the distal end of the delivery device so as to simultaneously extend the anchor in the deployed configuration through the native valve from the first side to a second side of the native valve and 10 rotate the anchor in the deployed configuration around native chordae.

### CROSS REFERENCE TO RELATED APPLICATIONS

This application claims priority to U.S. Provisional Patent Application No. 63/072,797, filed on Aug. 31, 2021, titled “PROSTHETIC CARDIAC VALVE DELIVERY SYSTEMS AND METHODS,”, the entirety of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transeptally, requiring transapical access instead.

Thus, a new valve delivery system or method that overcomes some or all of these deficiencies is therefore desired.

### SUMMARY OF THE DISCLOSURE

In general, in one embodiment, a method for treating a diseased native valve in a patient includes advancing a distal end of a delivery device to a first side of a native valve, deploying an anchor from a delivery configuration to a deployed configuration on the first side of the native valve, and rotating the distal end of the delivery device so as to simultaneously extend the anchor in the deployed configuration through the native valve from the first side to a second side of the native valve and rotate the anchor in the deployed configuration around native chordae.

In general, in another embodiment, a delivery device can have a distal end. The delivery device can be configured to advance to a first side of the native valve, deploy an anchor from a delivery configuration to a deployed configuration on the first side of the native valve, and rotate at the distal end so as to simultaneously extend the anchor in the deployed configuration through the native valve from the first side to a second side of the native valve and rotate the anchor in the deployed configuration around native chordae.

These and other embodiments can include one or more of the following features. The distal end of the delivery device can include an anchor guide having a preset curvature. The step of rotating can include rotating the anchor guide relative to an outer sheath of the delivery device. The anchor guide can be configured to place torsion on the anchor as the anchor transitions from the delivery configuration to the deployed configuration. Deploying the anchor can include deploying the anchor concentrically about the delivery device. Rotating the distal end can include rotating the anchor concentrically around the delivery device. The anchor can include a flat spiral in the deployed configuration. The method can further include aligning a plane of the flat spiral such that it is parallel with a plane of the mitral valve prior to the step of rotating the distal end. The step of aligning can be performed by steering an outer sheath of the delivery device. The step of aligning can be performed by translating or rotating an anchor guide of the delivery device, the anchor guide having a preset curvature. The anchor can be substantially straight within the delivery device in the delivery configuration. The method can further include expanding a valve prosthesis within the anchor when the anchor is in the deployed configuration around the native chordae. Expanding the valve prosthesis can include expanding the valve prosthesis such that a first end extends on the first side of the native valve and a second end extends on the second side of the native valve. The native valve can be a mitral valve, the first side of the native valve can include a left atrium, and the second side of the native valve can include a left ventricle. The method can further include transeptally inserting the distal end of the delivery device into a left atrium of the heart. Deploying the anchor can include pushing the anchor out of the delivery device. The method can further include releasing the anchor from the delivery device after an entirety of the anchor is on the second side of the valve and not on the first side of the valve. The method can further include extending a distal tip of the anchor through the valve at a commissure prior to the step of rotating the distal end. The method can further include confirming a position of the anchor with fluoroscopy or echocardiography. The delivery device can include a steerable outer sheath. The method can further include directing the distal end to a desired position with the steerable outer sheath. The method can further include translating the distal end of the delivery device towards the second side after the step of deploying and before the step of rotating.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1G show an exemplary supra-valvular method of delivering a spiral anchor to a mitral valve.

FIG. 2A shows exemplary alignment of an anchor prior to extending the anchor through the valve and around the chordae.

FIG. 2B shows an image of proposed anchor tip placement during a supra-valvular delivery.

FIGS. 3A-3G show another exemplary supra-valvular method of delivering a spiral anchor to a mitral valve.

### DETAILED DESCRIPTION

Described herein are methods and devices for use in delivering a spiral anchor, for example during a mitral valve replacement. In some embodiments, the spiral anchor can be part of a valve prosthesis that includes a frame and an anchor. The frame can include an expandable stent-like structure with a central lumen having leaflets. The frame can be configured to be placed within a diseased native valve to replace the diseased native valve. The anchor can have a spiral shape that is configured to encircle an outer perimeter of the frame. The anchor can provide an opposing force against the frame to anchor the frame with respect to native valve.

In some embodiments, the anchor can be deployed within the heart separately from the frame. During deployment, the anchor may extend from a catheter (e.g., that is part of an anchor deployment catheter system) to transition from a substantially straight configuration to a deployed spiral-shaped configuration. Once deployed within the heart, the position of the anchor can be manipulated to wrap the anchor around the chordae tendineae and/or native valve leaflets.

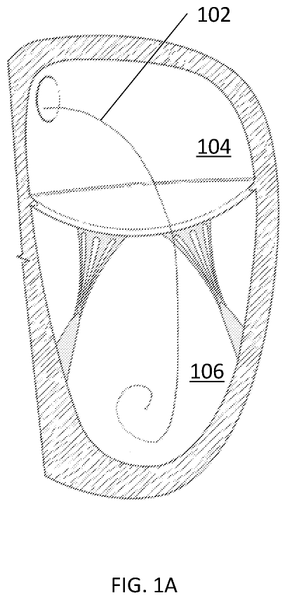

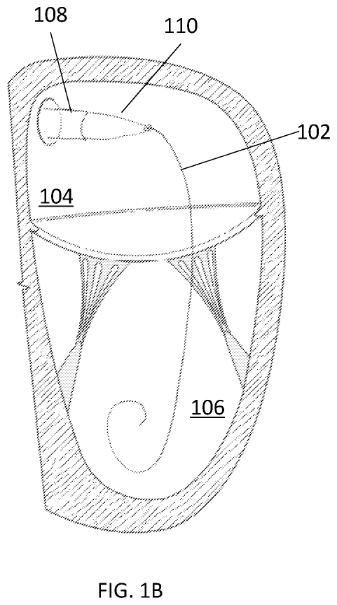

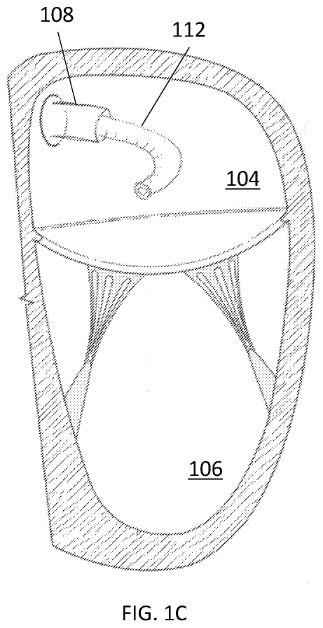

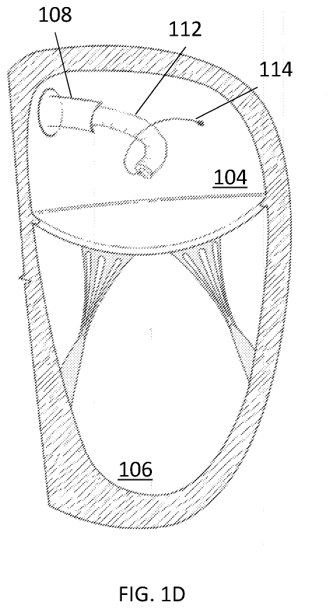

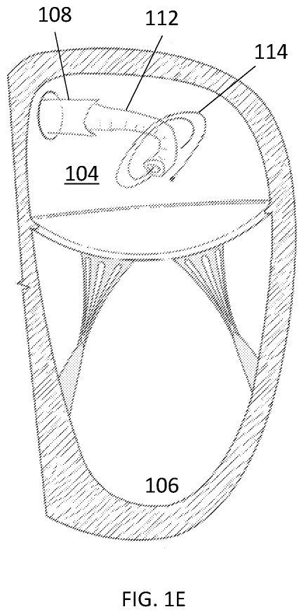

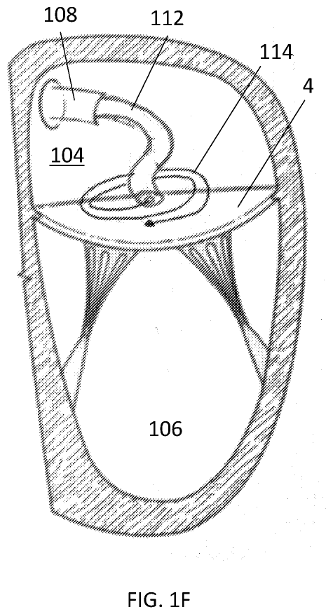

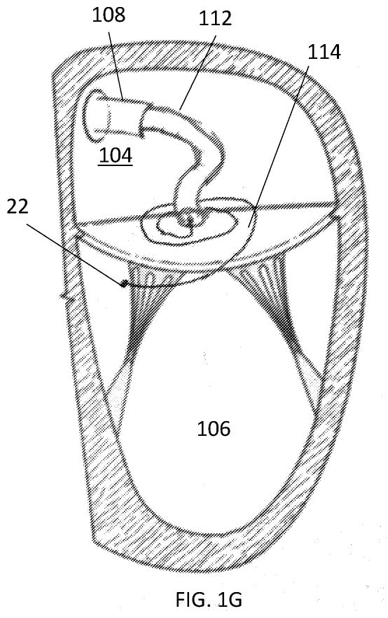

FIGS. 1A-1G show a supra-valvular method of delivering an anchor of a valve system. At FIG. 1A, a transseptal puncture is made in the heart. The guidewire 102 is then routed through the puncture site and left either in the left atrium 104 or across the mitral valve into the left ventricle 106. At FIG. 1B, an outer sheath 108 (optionally with an inner dilator 110) is tracked over the guidewire 102 until the distal end of the outer sheath 108 protrudes into the left atrium 104. The guidewire 102 and inner dilator 110 are then removed from the outer sheath 108. At FIG. 1C, an inner sheath and anchor guide 112 are inserted through the outer sheath 108 until the distal tip of the anchor guide 112 extends into the left atrium 104. The anchor guide 112 can be positioned and/or oriented as desired by steering the distal end of the sheath 108 and/or rotating the inner shaft and anchor guide 112 within the sheath 108. At FIG. 1D, once the anchor guide 112 is in the correct orientation, the anchor 114 can be pushed out through distal tip of the anchor guide 112. At FIG. 1E, the curvature of the anchor guide 112 can cause torsion on the anchor 114, causing the anchor 114 to deploy concentrically with the outer sheath 108 into the atrium 104. At FIG. 1F, the anchor 114 is oriented (e.g., via steering of the outer sheath 108 or movement or steering of the anchor guide 112) such that the plane of the spiral of the anchor 114 is parallel to the plane of the mitral valve 4. At FIG. 1G, the anchor tip 22 is extended through the valve at a commissure, and the anchor 114 is rotated (e.g., via rotation of the anchor guide 112) such that anchor 114 begins to encircle the mitral leaflets and chordae. The anchor 114 can then be rotated further until the anchor 114 fully encircles the mitral leaflets and chordae and extends entirely within the ventricle 106. After the anchor 114 has been positioned, a prosthetic valve can be positioned (e.g., expanded) therein. In some embodiments, a portion of the prosthetic valve can extend into the atrium and a portion into the ventricle. The anchor guide 112 and/or sheath 108 can be removed from the heart either before or after the prosthetic valve is positioned within the anchor 114.

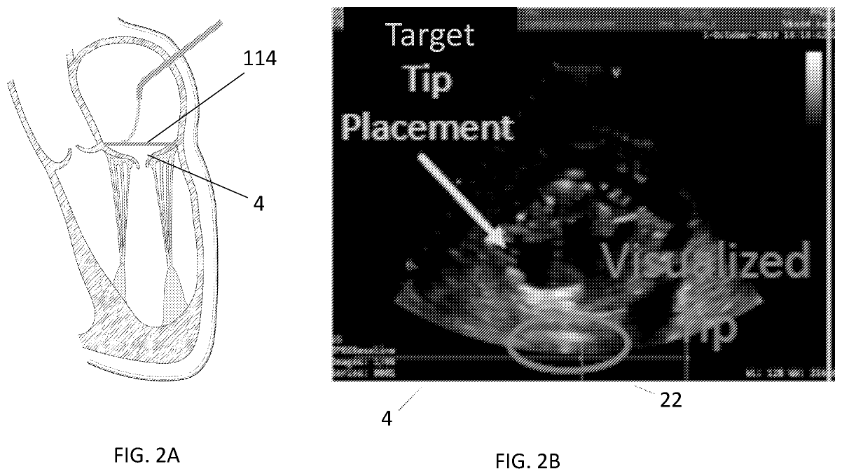

As shown in FIGS. 2A-2B, fluoroscopy and/or echocardiography can be used to ensure that the anchor 114 is parallel with the annulus of the mitral valve 4 and/or to ensure that the distal tip 22 of the anchor 114 extends through the mitral valve 4 at a commissure.

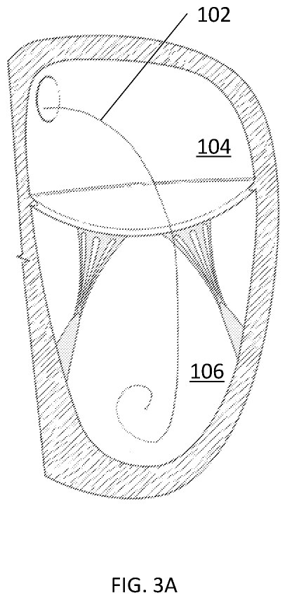

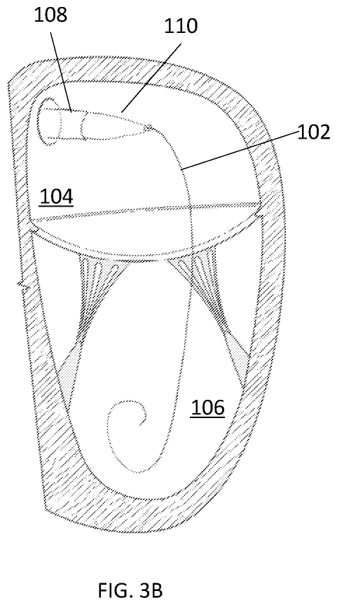

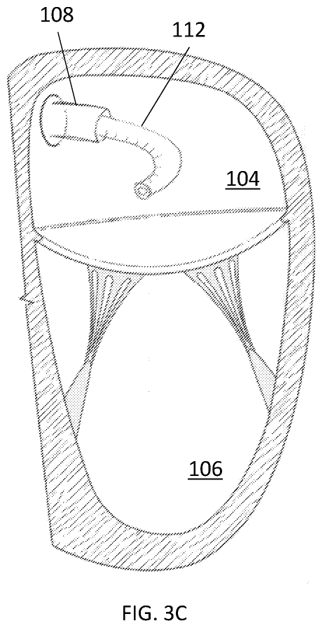

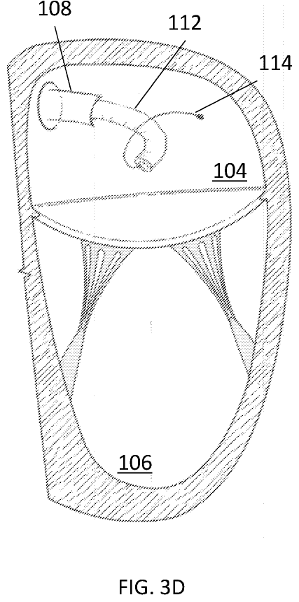

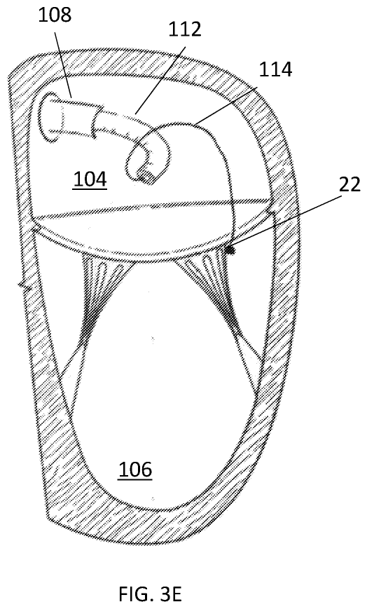

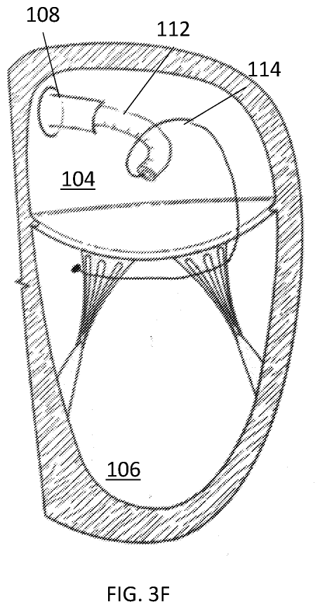

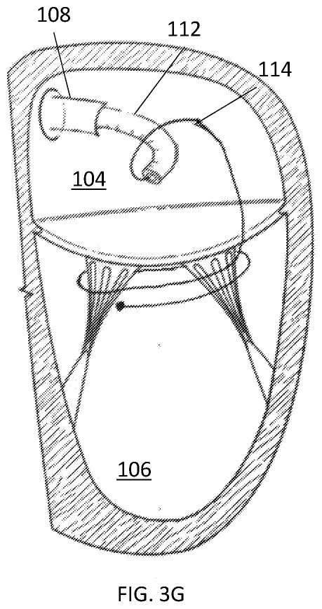

Another exemplary supra-valvular delivery method is shown in FIGS. 3A-3G. The delivery method of FIGS. 3A-3G is similar to the method of FIGS. 1A-1G except that the anchor 114 is extended through the commissures and rotated at least partially around the chordae before the anchor 114 is fully extended out of the anchor guide 112. Thus, at FIG. 3A, a transseptal puncture is made in the heart. The guidewire 102 is then routed through the puncture site and left either in the left atrium 104 or across the mitral valve into the left ventricle 106. At FIG. 3B, an outer sheath 108 (optionally with an inner dilator 110) is tracked over the guidewire 102 until the distal end of the outer sheath 108 protrudes into the left atrium 104. The guidewire 102 and inner dilator 110 are then removed from the outer sheath 108. At FIG. 3C, an inner sheath and anchor guide 112 are inserted through the outer sheath 108 until the distal tip of the anchor guide 112 extends into the left atrium 104. The anchor guide 112 can be positioned and/or oriented as desired by steering the distal end of the sheath 108 and/or rotating the inner shaft and anchor guide 112 within the sheath 108. In some embodiments, rather than aligning the plane of the anchor 114 with the plane of the mitral valve 4 (as described with respect to FIGS. 1A-1G), the tip of the anchor guide 112 can be aligned with the plane of the mitral valve 4. At FIG. 3D, once the anchor guide 112 is in the correct orientation, the anchor 114 can begin to be pushed out through distal tip of the anchor guide 112. At FIG. 3E, the distal tip 22 of the anchor 114 can be pushed further out of the anchor guide 112 and extended through the valve at a commissure. In some embodiments, steering of the sheath 108 and/or rotation of the guide 112 can assist in placement of the distal tip 22 at the commissure. At FIG. 3F, the anchor 114 can be extended further out of the sheath 108 and wrapped around the mitral leaflets and chordae. In some embodiments, rotating of the guide 112 can assist in wrapping the anchor 114 around the commissures. At FIG. 3G, the anchor 114 can be extended until the anchor 114 fully encircles the mitral leaflets and chordae and is positioned entirely within the ventricle 106. After the anchor 114 has been positioned, a prosthetic valve can be positioned (e.g., expanded) therein. In some embodiments, a portion of the prosthetic valve can extend into the atrium and a portion into the ventricle. The anchor guide 112 and/or sheath 108 can be removed from the heart either before or after the prosthetic valve is positioned within the anchor 114.

Advantageously, the supra-valvular delivery methods described herein can reduce initial anchor alignment requirements (because the anchor tip 22 can begin encircling even without the plane of the anchor 114 be perfectly aligned with the plane of the mitral valve 4 provided that the tip 22 is placed at the commissure), allow the mitral valve 4 to fully close during encircling (because only the anchor 114 extends through the valve), and/or minimize the interaction between the anchor 114 and delivery system with the sub-valvular anatomy.

In some embodiments, the anchor and the valve prosthesis are delivered into the heart using a single catheter delivery system. In other embodiments, the anchor and valve prosthesis are delivered into the heart in separate catheter delivery systems. In some embodiments, a tether may hold a proximal end of the anchor after delivery of the anchor. The tether may, in some embodiments, be used to adjust a position of the anchor. Further, the tether may, in some embodiments, enable passage of the valve prosthesis and/or valve delivery device thereover. Embodiments of methods and devices for delivering an anchor/valve prosthesis are described in U.S. patent application Ser. No. 16/824,576, filed on Mar. 19, 2020, titled “PROSTHETIC CARDIAC VALVE DEVICES, SYSTEMS, AND METHODS,” in U.S. patent application Ser. No. 16/594,946, filed on Oct. 7, 2019, titled “PROSTHETIC CARDIAC VALVE DEVICES, SYSTEMS, AND METHODS,” in International Application No. PCT/US2021/026463, filed Apr. 8, 2021, titled “VALVE DELIVERY SYSTEM,” and in International Application No. PCT/US2021/040623, filed on Jul. 7, 2021, titled “VALVE DELIVERY SYSTEM,” the entire disclosures of which are incorporated by reference herein.

As described herein, the anchor 114 may comprise a flat spiral shape with a plurality of windings or loops spiraling radially outwards from a central point. The loops of the flat spiral shaped anchor may be generally positioned within the same plane. The anchor may be formed from a shape memory material (e.g., NiTi). The anchor can be configured to extend around the chordae of the valve (e.g., the mitral valve) and around the valve prosthesis to hold the valve prosthesis in place. Flat spiral anchors are described in U.S. patent application Ser. No. 16/723,537, filed on Dec. 20, 2019, titled “PROSTHETIC CARDIAC VALVE DEVICES, SYSTEMS, AND METHODS,” now U.S. Publication No. US-2020-0261220-A1, the entirety of which is incorporated by reference herein.

As described herein, the valve prosthesis may include a frame structure (e.g., with a tapered waist and atrial and ventricular flared portions). The valve segment may include a plurality (e.g., three) leaflets. In some embodiments, the leaflets can be formed of multi-layered materials for preferential function. The valve segment may be attached directly to the frame structure. Alternatively, the valve segment may be attached to an intermediate valve structure that is in turn connected to the frame structure. The valve segment may be connected to the frame structure before or after the frame structure has been deployed adjacent a native valve. The valve segment may comprise a biocompatible one-way valve. Flow in one direction may cause the leaflets to deflect open and flow in the opposite direction may cause the leaflets to close. The frame structure may be configured like a stent. The frame structure may, for example, comprise a scaffold in a diamond pattern formed from a shape memory material (e.g., nitinol, NiTi). One of ordinary skill in the art will appreciate that many other structures, materials, and configurations may be employed for the frame structure. For example, the frame structure may be formed of a polymer of sufficient elasticity. The frame structure may be formed of a combination of metal and polymer, such as metal (e.g., shape memory material) covered in polymer. The frame structure may include a variety of patterns besides diamond shapes. In some embodiments, the frame structure is a closed frame such that blood flow is forced through valve segment therein. One or more skirts and/or seals may help force blood through the valve segment. Exemplary frame structures and valve prostheses are described in International Patent Application No. PCT/US2020/027744, filed on Apr. 10, 2020, titled “MINIMAL FRAME PROSTHETIC CARDIAC VALVE DELIVERY DEVICES, SYSTEMS, AND METHODS,” now PCT Publication No. WO 2020/210685, and International Patent Application No. PCT/US2021/037661, filed on Jun. 16, 2021, titled “MINIMAL FRAME PROSTHETIC CARDIAC VALVE DELIVERY DEVICES, SYSTEMS, AND METHODS,” the entireties of which are incorporated by reference herein.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

A complete listing of the claims follows:

1. A method for treating a diseased native valve in a patient, the method comprising:
advancing a distal end of a delivery device to a first side of a native valve;
deploying an anchor from a delivery configuration to a deployed configuration on the first side of the native valve; and
rotating the distal end of the delivery device so as to simultaneously extend the anchor in the deployed configuration through the native valve from the first side to a second side of the native valve and rotate the anchor in the deployed configuration around native chordae.

2. The method of claim 1, wherein the distal end of the delivery device comprises an anchor guide having a preset curvature, and wherein the step of rotating comprises rotating the anchor guide relative to an outer sheath of the delivery device.

3. The method of claim 2, wherein the anchor guide is configured to place torsion on the anchor as the anchor transitions from the delivery configuration to the deployed configuration.

4. The method of claim 1, wherein deploying the anchor comprises deploying the anchor concentrically about the delivery device.

5. The method of claim 1, wherein rotating the distal end comprises rotating the anchor concentrically around the delivery device.

6. The method of claim 1, wherein the anchor comprises a flat spiral in the deployed configuration.

7. The method of claim 6, further comprising aligning a plane of the flat spiral such that it is parallel with a plane of the mitral valve prior to the step of rotating the distal end.

8. The method of claim 7, wherein the step of aligning is performed by steering an outer sheath of the delivery device.

9. The method of claim 7, wherein the step of aligning is performed by translating or rotating an anchor guide of the delivery device, the anchor guide having a preset curvature.

10. The method of claim 1, wherein the anchor is substantially straight within the delivery device in the delivery configuration.

11. The method of claim 1, further comprising expanding a valve prosthesis within the anchor when the anchor is in the deployed configuration around the native chordae.

12. The method of claim 11, wherein expanding the valve prosthesis comprises expanding the valve prosthesis such that a first end extends on the first side of the native valve and a second end extends on the second side of the native valve.

13. The method of claim 1, wherein the native valve is a mitral valve, the first side of the native valve comprises a left atrium, and the second side of the native valve comprises a left ventricle.

14. The method of claim 1, further comprising transeptally inserting the distal end of the delivery device into a left atrium of the heart.

15. The method of claim 1, wherein deploying the anchor comprises pushing the anchor out of the delivery device.

16. The method of claim 1, further comprising releasing the anchor from the delivery device after an entirety of the anchor is on the second side of the valve and not on the first side of the valve.

17. The method of claim 1, further comprising extending a distal tip of the anchor through the valve at a commissure prior to the step of rotating the distal end.

18. The method of claim 1, further comprising confirming a position of the anchor with fluoroscopy or echocardiography.

19. The method of claim 1, wherein the delivery device comprises a steerable outer sheath, the method further comprising directing the distal end to a desired position with the steerable outer sheath.

20. The method of claim 1, further comprising translating the distal end of the delivery device towards the second side after the step of deploying and before the step of rotating.

21. (canceled)
